(19) United States
(12) Patent Application Publication
(10) Pub. No.: US 2025/0228501 A1
(43) Pub. Date: Jul. 17, 2025

(54) SYSTEMS AND METHODS OF PREDICTING AND MANAGING BLOOD GLUCOSE LEVELS IN INDIVIDUALS

(71) Applicant: Koninklijke Philips N.V., Eindhoven (NL)

(72) Inventors: LIEKE GERTRUDA ELISABETH COX, Eindhoven (NL); Gabriele Papini, Eindhoven (NL); Jens Muehlsteff, Eindhoven (NL); Eduard Gerard Marie Pelssers, Eindhoven (NL); Niel Francis Joyce, Best (NL)

(21) Appl. No.: 19/017,898

(22) Filed: Jan. 13, 2025

Related U.S. Application Data
(60) Provisional application No. 63/620,230, filed on Jan. 12, 2024.

(51) Int. Cl. A61B 5/00; A61B 5/0205; A61B 5/145; G16H 20/00

(52) U.S. Cl. CPC A61B 5/7275; A61B 5/0205; A61B 5/7267; G16H 20/00; A61B 5/14532

(57) ABSTRACT

Provided herein are systems and methods of predicting and managing blood glucose levels in individuals, including systems and methods of predicting blood glucose levels based on predicted future glucose factors. Also provided herein are systems and methods of recommending glucose interventions based thereon. It is appreciated by the present disclosure that it is better to prevent extreme blood glucose levels before they occur than merely detecting such levels when they occur. Accordingly, the systems and methods described herein utilized a combination of contextual information and current time glucose measurements/estimates to predict the likelihood of different scenarios that might lead to such extreme levels before they occur.

### FIELD OF THE DISCLOSURE

The present disclosure relates generally to monitoring glucose levels of one or more individuals, and more specifically to systems and methods of predicting and managing blood glucose levels of individuals.

### BACKGROUND

Monitoring blood glucose levels is important for individuals with diabetes, a chronic condition where the body struggles to regulate blood sugar effectively. People diagnosed with diabetes need to monitor their blood glucose levels regularly; however, it should be appreciated that other individuals, such as those at risk of developing diabetes, may also desire to monitor their blood glucose levels to ensure that they remain in an acceptable range (i.e., avoiding hyperglycemic and hypoglycemic states). In conventional glucose monitoring situations, an individual may actively self-monitor using a finger prick and blood analysis device, or may use a continuous glucose monitoring device that continuously measures the individual's blood glucose levels. Then, depending on the results of the current time measurements, the individual may decide to take a particular action, such as, for example, taking a prescribed dosage of insulin or cating a snack.

### SUMMARY OF THE DISCLOSURE

It is recognized by the present disclosure that monitoring blood glucose levels is very important as it helps individuals manage their diabetes effectively. By maintaining blood glucose within an acceptable range, individuals can prevent complications like heart disease, stroke, kidney disease, and nerve damage, among others. However, Applicant appreciated that it is better to predict and prevent extreme blood glucose levels than merely detecting such levels once reached. Accordingly, the present disclosure is directed to systems and methods of improving the accuracy and usefulness of blood glucose predictions.

According to an embodiment of the present disclosure, a system for managing glucose levels of an individual is provided. The system can include: one or more data sources configured to generate contextual information associated with an individual; a glucose measuring device configured to generate at least one glucose measurement for the individual; a non-transitory computer-readable storage medium having stored thereon computer-readable instructions; and one or more processors in communication with the computer-readable storage medium, wherein the one or more processors are configured by the computer-readable instructions stored thereon to perform the following operations: (i) obtain, from the one or more data sources, contextual information associated with the individual; (ii) predict, using a first trained model, one or more future glucose factors for the individual based on the contextual information received, wherein the one or more future glucose factors are relevant to at least one future time period; (iii) obtaining, from the glucose measuring device, at least one current glucose measurement for the individual; (iv) predict, using a second trained model, a future glucose indication for the individual within the at least one future time period based on the one or more future glucose factors predicted and the at least one glucose measurement obtained for the individual; (v) provide, via a user device, the future glucose indication predicted for the individual.

In an aspect, the one or more processors may be further configured to perform the following operations: (vi) generate a glucose intervention for the individual based on the future glucose indication predicted for the individual within the at least one future time period; and (vii) provide, via the user device, the glucose intervention generated for the individual and the future glucose indication predicted for the individual.

In an aspect, the one or more data sources may include at least one of: a digital calendar of the individual; an email account; a social media account; and a text messaging account.

In an aspect, the first trained model may include a natural language processor trained to extract future glucose factors from the contextual information received, the future glucose factors comprising anticipated meal consumption, anticipated physical activity, anticipated rest, anticipated alcohol consumption, and/or anticipated stress levels.

In an aspect, the one or more future glucose indications predicted for the individual may be conditioned on a likelihood of one or more future glucose factors predicted for the individual.

In an aspect, the glucose measuring device may also be the user device.

In an aspect, the system may further include a user device configured to provide to the individual glucose interventions generated for the individual and the future glucose indications predicted for the individual.

According to another embodiment of the present disclosure, a method of predicting a future glucose indication of an individual is provided. The method can include: (i) obtaining, from one or more data sources, contextual information associated with the individual; (ii) predicting, using a first trained model, one or more future glucose factors for the individual based on the contextual information received, wherein the one or more future glucose factors are relevant to at least one future time period; (iii) obtaining, from a glucose measuring device, at least one current glucose measurement for the individual; and (iv) predicting, using a second trained model, the future glucose indication for the at least one future time period based on the one or more future glucose factors predicted and the at least one current glucose measurement obtained for the individual.

In an aspect, the one or more future glucose indications predicted for the individual may be conditioned on a likelihood of one or more future glucose factors predicted for the individual.

In an aspect, the one or more data sources may include at least one of: a photoplethysmography device; an electrocardiogram device; a galvanic skin response device; an accelerometer; a barometer; a gyroscope; a microphone; a camera; a global positioning system (GPS); and a thermometer.

In an aspect, the one or more data sources may include at least one of: a digital calendar of the individual; an email account; a social media account; and a text messaging account.

In an aspect, the first trained model may include a natural language processor trained to extract future glucose factors from the contextual information received, the future glucose factors comprising anticipated meal consumption, anticipated physical activity, anticipated rest, anticipated alcohol consumption, and/or anticipated stress levels.

In an aspect, the one or more data sources may include a user input device, and the contextual information may include user input received via the user input device.

In an aspect, the at least one future time period may include a time period encompassing between 1 and 2 hours in the future from a current time period.

In an aspect, the second trained model may include a biophysical model, a transfer function, and/or an artificial intelligence model trained to predict one or more future glucose indications of a subject based on one or more current glucose level measurements obtained for the subject.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a block diagram illustrating a system for recommending a glucose intervention based on a predicted future glucose indication in accordance with aspects of the present disclosure.

FIG. 2 is a block diagram illustrating a prediction and recommendation module configured to predict future glucose indications of an individual and recommend glucose interventions in accordance with aspects of the present disclosure.

FIG. 3A is a flowchart illustrating a method of predicting a future glucose indication of an individual in accordance with aspects of the present disclosure.

FIG. 3B is a flowchart illustrating a method of predicting a future glucose indication of an individual in accordance with further aspects of the present disclosure.

### DETAILED DESCRIPTION OF EMBODIMENTS

As mentioned above, monitoring blood glucose levels is very important for individuals with diabetes as it helps maintain blood glucose levels within an acceptable range, thereby preventing complications like heart disease, stroke, kidney disease, and nerve damage, among others. Furthermore, it is desirable to prevent extreme blood glucose levels (i.e., too high or too low) rather than simply detecting such states. While past glucose measurements may be used to try and predict upcoming changes in blood glucose levels, these approaches do not account for a number of factors that could potentially impact future glucose levels, such as anticipated meal consumption, anticipated physical activity, anticipated sleep/rest, anticipated alcohol consumption, anticipated stress levels, and/or the like. Accordingly, provided herein are systems and methods of improving the accuracy and usefulness of blood glucose predictions by also predicting such future glucose factors.

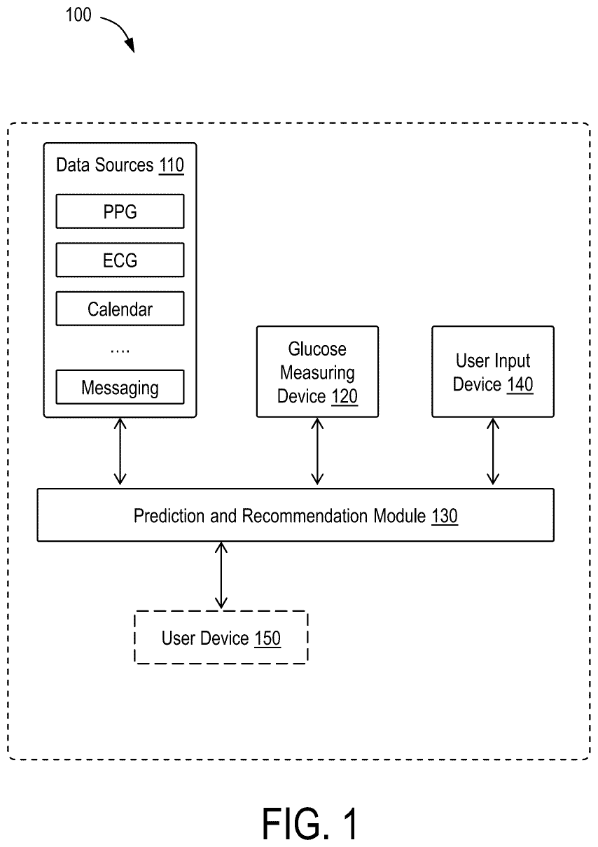

For example, with reference to FIG. 1, a system 100 adapted to predict one or more future glucose indications of an individual and recommend an intervention based on a predicted future glucose indication is illustrated in accordance with various aspects of the present disclosure. As shown, the system 100 can include one or more data sources 110 and a glucose measuring device 120 in communication with a prediction and recommendation module 130.

In embodiments, the data sources 110 can include one or more types of data sources that are configured to generate contextual information associated with an individual undergoing blood glucose monitoring. In certain embodiments, the data sources 110 can include one or more of the following: a photoplethysmography device; an electrocardiogram device; a galvanic skin response device; an accelerometer; a barometer; a gyroscope; a microphone; a camera; a global positioning system (GPS); and a thermometer. In further embodiments, the data sources 110 can include one or more of the following: a digital calendar; an email account; a social media account; and a text messaging account.

In particular embodiments, the system 100 may also include a user input device 140 configured to receive contextual information from an individual through direct user interaction. That is, an individual may utilize a user input device 140 to manually provide contextual information. In some embodiments, for example, the individual may utilize the user input device 140 to input contextual information in response to one or more prompts by the system 100. The user input device 140 can include, but is not limited to, a keyboard, keypad, trackpad, trackball(s), capacitive keyboard, controller (e.g., a gaming controller), computer mouse, computer stylus/pen, a voice input device, and/or the like, including combinations thereof.

As described herein, contextual information can include text, images, audio, video, sensor measurements, location data, and/or other forms of data that can provide insight into one or more future glucose factors. For example, in embodiments, future glucose factors can include anticipated meal consumption, anticipated physical activity, anticipated sleep/rest, anticipated alcohol consumption, anticipated stress levels, and/or the like. Thus, contextual information relating to one or more of these future glucose factors may be mined from the one or more data sources 110.

In embodiments, the glucose measuring device 120 can include one or more types of glucose monitoring devices or kits configured to measure/estimate current blood glucose levels for an individual. For example, in embodiments, the glucose measuring device 120 can include a finger prick device, a continuous glucose monitor, a photoplethysmography device, an electrocardiogram device, a galvanic skin response device, and/or the like.

In embodiments, the glucose prediction and recommendation module 130 can be configured to perform one or more steps of the methods described herein. In particular, the glucose prediction and recommendation module 130 can be configured to predict a future glucose indication of an individual based on information/data received from the data sources 110, the glucose measuring device 120, and one or more other devices (e.g., a user input device 140). The future glucose indication can include, but is not limited to, a future glucose level, and/or a range of future glucose levels. In some embodiments, the future glucose indication can also include a confidence interval for a predicted glucose level or glucose range. In some embodiments, the glucose prediction and recommendation module 130 may be configured to predict a plurality of future glucose indications, such as multiple future glucose levels with different confidence intervals that account for different contextual information (e.g., likely glucose levels if the subject remains sedentary for the next hour versus if the subject takes a walk, etc.). In further embodiments, the glucose prediction and recommendation module 130 can also be configured to generate a recommendation intended to prevent extreme blood glucose level states for the individual (e.g., hyperglycemia or hypoglycemia).

In particular embodiments, the glucose prediction and recommendation module 130 can include one or more processors 202 and a computer-readable memory 204 interconnected and/or in communication via a system bus 206 containing conductive circuit pathways through which instructions (e.g., machine-readable signals) may travel to effectuate communication, tasks, storage, and the like. The glucose prediction and recommendation module 130 can be connected to a power source (not shown), which can include an internal power supply and/or an external power supply. In embodiments, the glucose prediction and recommendation module 130 can also include one or more additional components, such as a display 210, an input/output (I/O) interface 212, a networking unit 214, and the like, including combinations thereof. As shown, each of these components may be interconnected and/or in communication via the system bus 206, for example.

In embodiments, the one or more processors 202 can include one or more high-speed data processors adequate to execute the program components described herein and/or perform one or more operations of the methods described herein. The one or more processors 202 may include a microprocessor, a multi-core processor, a multithreaded processor, an ultra-low voltage processor, an embedded processor, and/or the like, including combinations thereof. The one or more processors 202 can include multiple processor cores on a single die and/or may be a part of a system on a chip (SoC) in which the processor 202 and other components are formed into a single integrated circuit, or a single package. That is, the one or more processors 202 may be a single processor, multiple independent processors, or multiple processor cores on a single die.

In embodiments, the display device 210 may be configured to display information, including text, graphs, and/or the like. In particular embodiments, the display device 210 may be configured to provide a recommended glucose intervention to a user of the system 100. The display device 210 can include, but is not limited to, a liquid crystal display (LCD), a light-emitting diode (LED) display, a touch screen or other touch-enabled display, a foldable display, a projection display, and so on, or combinations thereof.

In embodiments, the input/output (I/O) interface 212 may be configured to connect and/or enable communication with one or more peripheral devices (not shown), including but not limited to additional machine-readable memory devices, diagnostic equipment, and other attachable devices. The I/O interface 212 may include one or more I/O ports that provide a physical connection to the one or more peripheral devices. In some embodiments, the I/O interface 212 may include one or more serial ports.

In embodiments, the networking unit 214 may include one or more types of networking interfaces that facilitate wired and/or wireless communication between the glucose prediction and recommendation module 130 and one or more external devices. That is, the networking unit 214 may operatively connect the glucose prediction and recommendation module 130 to one or more types of communications networks 216, which can include a direction interconnection, the Internet, a local area network (“LAN”), a metropolitan area network (“MAN”), a wide area network (“WAN”), a wired or Ethernet connection, a wireless connection, a cellular network, and similar types of communications networks, including combinations thereof. In some embodiments, the glucose prediction and recommendation module 130 may communicate with one or more remote/cloud-based servers and/or cloud-based services, such as remote server 218, via the communications network 216.

In embodiments, the memory 204 can be variously embodied in one or more forms of machine accessible and machine-readable memory. In some embodiments, the memory 204 includes a storage device (not shown), which can include, but is not limited to, a non-transitory storage medium, a magnetic disk storage, an optical disk storage, an array of storage devices, a solid-state memory device, and/or the like, as well as combinations thereof. The memory 204 may also include one or more other types of memory, such as dynamic random-access memory (DRAM), static random-access memory (SRAM), erasable programmable read-only memory (EPROM), electrically erasable programmable read-only memory (EEPROM), Flash memory, and/or the like, as well as combinations thereof. In embodiments, the memory 204 may include one or more types of transitory and/or non-transitory memory.

The glucose prediction and recommendation module 130 can be configured by software components stored in the memory 204 to perform one or more processes of the methods described herein. More specifically, the memory 204 can be configured to store data/information 220 and computer-readable instructions 222 that, when executed by the one or more processors 202, causes the glucose prediction and recommendation module 130 to perform the following operations: (i) obtain, from one or more data sources 110, contextual information associated with an individual; (ii) predict, using a first trained model, one or more future glucose factors for the individual based on the contextual information received, wherein the one or more future glucose factors are relevant to at least one future time period; (iii) obtain, from a glucose measuring device 120, at least one current glucose measurement for the individual; (iv) predict, using a second trained model, a future glucose indication for the individual within the at least one future time period based on the one or more future glucose factors predicted and the at least one glucose measurement obtained for the individual; (v) generate a glucose intervention for the individual based on the future glucose indication predicted for the individual within the at least one future time period; and (vi) provide, via a user device (e.g., user device 150), the glucose intervention generated for the individual and the future glucose indication predicted for the individual within the at least one future time period.

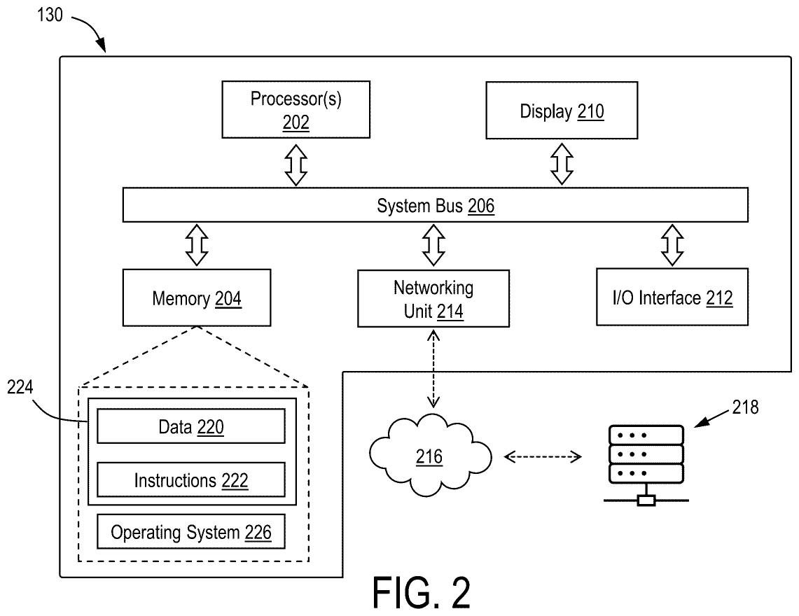

As shown in the example of FIG. 2, such data 220 and the computer-readable instructions 222 stored in the memory 204 may form a glucose prediction and recommendation package 224 that can be incorporated into, loaded from, loaded onto, or otherwise operatively available to and from the glucose prediction and recommendation module 130. In embodiments, this glucose prediction and recommendation package 224 can include one or more trained models, such as one or more trained artificial intelligence models.

In particular embodiments, the glucose prediction and recommendation package 224 includes at least a first trained model that is configured to determine the likelihood of one or more future glucose factors occurring for an individual. For example, the first trained model may include a natural language processor trained to review the contextual information and extract from the contextual information one or more future glucose factors, such as anticipated meal consumption, anticipated physical activity, anticipated rest, anticipated alcohol consumption, and/or anticipated stress levels.

In embodiments, the one or more future glucose factors may be relevant to at least one future time period, such as within the next six hours, twelve hours, twenty-four hours, forty-eight hours, and so on. Thus, for example, the first trained model may analyze the contextual information to predict the likelihood that an individual will consume a meal within at least one future time period (e.g., <1 hour, 1-2 hours, 2-4 hours, etc.), or will perform some physical activity within at least one future time period, or will go to sleep or rest within at least one future time period, or will consume alcohol within at least one future time period, or will have elevated stress levels within at least one future time period. Although certain future glucose factors are described herein, it should be appreciated that other factors may be included.

In further embodiments, the glucose prediction and recommendation package 224 can include at least a second trained model that is configured to predict one or more future glucose indications for the individual based on the future glucose factors and one or more current glucose measurements. In embodiments, each predicted future glucose indication can include a future glucose level, a range of future glucose levels, and/or a confidence interval for a predicted glucose level or range. Put another way, the second trained model may be configured to predict a future glucose level, a future glucose range, and/or a future glucose level confidence interval. The second trained model can include, but is not limited to, a biophysical model, a transfer function, and/or an artificial intelligence model, including combinations thereof.

In embodiments, the future glucose indication may be predicted for at least one future time period, such as the future time period where the future glucose factors are relevant. Put another way, the second trained model can be configured to predict future glucose indications for at least the similar future time period(s) as the first trained model. For example, if the first trained model predicted a significant likelihood that an individual will go out to dinner within the next three hours, the second trained model may predict a blood glucose level for the individual following that meal likely to occur within the next three hours.

In some embodiments, the glucose prediction and recommendation package 224 and/or one or more individual software packages (e.g., the trained models, etc.) may be stored in a local storage device of the memory 204. However, in other embodiments, the glucose prediction and recommendation package 224 and/or one or more individual software packages may be loaded onto and/or updated from a remote server or service, such as server 218, via the communications network 216.

The glucose prediction and recommendation module 130 may also include an operating system component 226, which may be stored in the memory 204. The operating system component 224 may be an executable program facilitating the operation of the system 100. Typically, the operating system component 226 can facilitate access of the I/O interface 212, network interface 214, the user interface 208, and the display 210, and can communicate or control other components of the system 100.

Accordingly, provided herein is a computer program product 224 comprising a non-transitory computer-readable storage medium 204 having stored thereon computer-readable instructions 222 that, when executed by one or more processors (such as processors 202), cause the one or more processors to perform one or more operations of the methods described herein.

For example, in specific embodiments, the computer-readable storage medium 204 may include computer-readable instructions 222 that, when executed by one or more processors (such as processors 202), cause the one or more processors to perform the following operations: (i) obtain, from one or more data sources 110, contextual information associated with an individual; (ii) predict, using a first trained model, one or more future glucose factors for the individual based on the contextual information received, wherein the one or more future glucose factors are relevant to at least one future time period; (iii) obtain, from a glucose measuring device 120, at least one current glucose measurement for the individual; (iv) predict, using a second trained model, a future glucose indication for the individual within the at least one future time period based on the one or more future glucose factors predicted and the at least one glucose measurement obtained for the individual; (v) generate a glucose intervention for the individual based on the future glucose indication predicted for the individual within the at least one future time period; and (vi) provide, via a user device (e.g., user device 150), the glucose intervention generated for the individual and the future glucose indication predicted for the individual within the at least one future time period

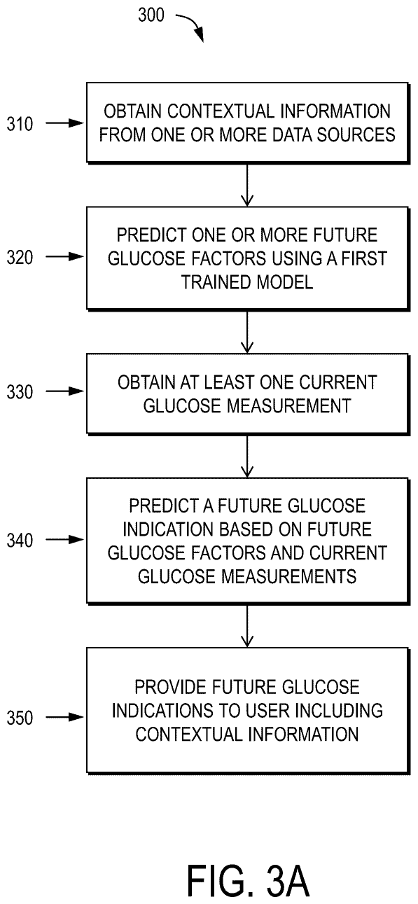

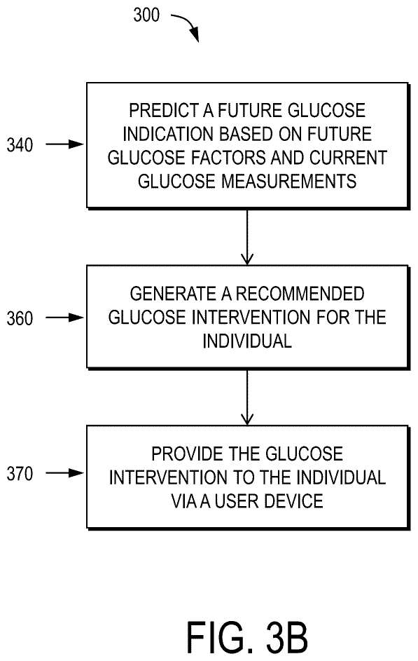

In particular embodiments, with reference to FIG. 3A, a method 300 for predicting a future glucose indication of an individual is illustrated in accordance with certain aspects of the present disclosure. As shown, the method 300 can include: in a step 310, obtaining contextual information associated with the individual; in a step 320, predicting one or more future glucose factors for the individual based on the contextual information received; in a step 330, obtaining at least one current glucose measurement for the individual; and in a step 340, predicting a future glucose indication based on the one or more future glucose factors predicted and the at least one current glucose measurement obtained for the individual.

More specifically, the step 310 of the method 300 can include obtaining contextual information associated with an individual from one or more data sources 110. As described above, contextual information can include, but is not limited to, text, images, audio, video, sensor measurements, location data, and/or other forms of data that can provide insight into one or more future glucose factors (e.g., anticipated meal consumption, anticipated physical activity, anticipated sleep/rest, anticipated alcohol consumption, anticipated stress levels, and/or the like).

In embodiments, the data sources 110 can include one or more of the following: a photoplethysmography device; an electrocardiogram device; a galvanic skin response device; an accelerometer; a barometer; a gyroscope; a microphone; a camera; a global positioning system (GPS); and a thermometer. As such, the contextual information can be, for example, PPG measurements, ECG measurements, GSR measurements, motion measurements, pressure measurements, rotation measurements, sounds, images, tracking information, temperature measurements, and/or the like, including combinations thereof.

In particular embodiments, the data sources 110 include one or more of the following: a digital calendar; an email account; a social media account; and a text messaging account. As such, the contextual information can include text or images from a digital calendar, an email account, a social media account, a text messaging account, and/or the like, including combinations thereof.

In further embodiments, the data sources 110 can also include a user input device 140 configured to receive contextual information from an individual through direct user interaction. In embodiments, the user input device 140 can include, but is not limited to, a keyboard, keypad, trackpad, trackball(s), capacitive keyboard, controller (e.g., a gaming controller), computer mouse, computer stylus/pen, a voice input device, and/or the like, including combinations thereof.

In some embodiments, the contextual information may be obtained by a prediction and recommendation module 130 using the data sources 110 and/or the user input device 140 as shown in the example of FIG. 1. The contextual information may be stored in the memory 204 as data 220, and/or may be communicated to a remote data storage server 218 via a communications network 216, as shown in the example of FIG. 2.

Next, in a step 320, the method 300 can include predicting one or more future glucose factors for the individual using at least a first trained prediction model. As described above, the first trained model can be configured to determine the likelihood of one or more future glucose factors occurring for an individual based on the contextual information received. In embodiments, the first trained model may include a natural language processor trained to review the contextual information and extract from the contextual information one or more future glucose factors, such as anticipated meal consumption, anticipated physical activity, anticipated rest, anticipated alcohol consumption, and/or anticipated stress levels.

In particular embodiments, the first trained model may also be configured to determine one or more characteristics or categories associated with a particular future glucose factor. In embodiments, these characteristics may be used to evaluate the impact of a particular future glucose factor. For example, if the first trained model predicts a high likelihood of an anticipated meal consumption, the first trained model may also categorize the anticipated meal based on additional contextual information (e.g., an unhealthy meal versus a healthy meal, etc.).

In embodiments, the one or more future glucose factors predicted by the first trained model may be relevant to at least one future time period, such as within the next one hour, one to two hours, two to four hours, and so on. Put another way, the first trained model may analyze the contextual information to predict the likelihood of a future glucose factor occurring within at least one future time period (e.g., <1 hours, 1-2 hours, 2-4 hours, etc.).

In a step 330, the method 300 can include obtaining at least one current glucose measurement for the individual. In embodiments, the current glucose measurements may be obtained using a glucose measuring device 120, such as a finger prick device, a continuous glucose monitor, a photoplethysmography device, an electrocardiogram device, a galvanic skin response device, and/or the like. As used herein, the term “current glucose measurement” refers to an actual real-time or near real-time measurement or estimation of an individual's blood glucose level as opposed to a predicted future value. In particular embodiments, the current glucose measurement may refer to an actual measurement or estimation obtained around the time that step 320 is executed. For example, in step 320, one or more future glucose factors are predicted with different likelihoods of occurring within the next 3-6 hours (i.e., a current time to plus 3-6 hours), then within the next several seconds or several minutes, one or more time glucose measurements are taken for the individual in the step 330.

In a step 340, the method 300 can then include predicting one or more future glucose indications for the individual based on the predicted future glucose factors and the current glucose measurements. In embodiments, the one or more future glucose indications may be predicted using at least a second trained model, which receives as inputs the predicted future glucose factors and the current glucose measurements. In some embodiments, the second trained model may also receive as inputs past glucose measurements as well as past contextual information. The second trained model can include, but is not limited to, a biophysical model, a transfer function, and/or an artificial intelligence model, including combinations thereof.

In particular embodiments, the one or more future glucose indications may be predicted in connection with the one or more future glucose factors. Put another way, the one or more future glucose indications predicted for the individual may be conditioned on a likelihood of one or more future glucose factors predicted for the individual. For example, if an anticipated meal consumption is predicted with a very high certainty within the next three hours, the second trained model may predict a future glucose indications for the individual that is conditioned on the anticipated meal consumption occurring. In another scenario, if an anticipated physical activity is predicted with a moderate certainty within the next four hours, the second trained model may predict a future glucose indication for the individual assuming the physical activity does occur and another future glucose indication for the individual assuming the physical activity does not occur. As such, the future glucose indications predicted may be generated such that they are relevant to the one or more future glucose factors and the at least one future time period.

In a step 350, the method 300 can then include providing one or more predicted glucose indications to a user via a user device 150. In particular embodiments, the predicted glucose indications may be provided to a user or subject of the device 120, or may be provided to a third-party such as a family member or healthcare specialist. In embodiments, the user device 150 can include, but is not limited to, a personal computer, laptop computer, mobile device, smartphone, tablet computer, gaming device, consumer electronic device, headset, smart watch, television, display monitor, patient monitor, and/or the like. As described above, the predicted glucose indications may include predicted glucose levels or ranges as well as confidence intervals for the prediction, all of which may be conditioned on one or more future glucose factors predicted. As such, the step 350 can also include providing one or more predicted glucose indications as well as the contextual information related to each particular predicted indication. For example, the system 100 may have identified a potential sedentary period for an individual and predicted that the individual's blood glucose levels increase to an undesirable level (e.g., the individual will become hyperglycemic), whereas if the individual takes a walk, then the hyperglycemic event will not occur. Accordingly, in the step 350, the predicted glucose indications (i.e., the glucose levels/ranges associated with taking a walk versus remaining sedentary) are provided to the user along with the associated glucose factors (i.e., sedentary versus walking).

With reference to FIG. 3B, the method 300 may further include: in a step 360, generating a recommended glucose intervention for the individual; and in a step 370, providing the glucose intervention to the individual via a user interface.

In particular embodiments, the step 360 can include generating a recommended glucose intervention based on the one or more predicted future glucose indications of the individual. In embodiments, the glucose intervention may be one or more types of alerts. In other embodiments, the glucose intervention may be a recommended action, such as a recommendation to prepare/take a particular dosage of insulin or prepare, have a meal or a snack, perform some level of exercise (e.g., take a walk), and/or the like. In embodiments, one or more glucose interventions may be generated in the step 360 that are conditioned on one or more future glucose factors. For example, two interventions may be generated in the case where the individual may or may not consume an unhealthy meal.

In the step 370, the method 300 can then include providing one or more glucose interventions to the individual via a user device 150, which can include, but is not limited to, a personal computer, laptop computer, mobile device, smartphone, tablet computer, gaming device, consumer electronic device, headset, smart watch, television, display monitor, patient monitor, and/or the like. In embodiments, the results of steps 310-350 may also be shown, for example, as a predicted glucose level profile with a confidence interval. In embodiments, the results provided via the user device 150 can be contextualized with the predicted glucose factors, e.g., when different scenarios would lead to levels requiring different interventions.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A system for managing glucose levels of an individual, the system comprising:
one or more data sources configured to generate contextual information associated with the individual;
a glucose measuring device configured to generate at least one glucose measurement for the individual;
a non-transitory computer-readable storage medium having stored thereon computer-readable instructions; and
one or more processors in communication with the computer-readable storage medium, wherein the one or more processors are configured by the computer-readable instructions stored thereon to perform the following operations: (i) obtain, from the one or more data sources, contextual information associated with the individual; (ii) predict, using a first trained model, one or more future glucose factors for the individual based on the contextual information received, wherein the one or more future glucose factors are relevant to at least one future time period; (iii) obtain, from the glucose measuring device, at least one current glucose measurement for the individual; (iv) predict, using a second trained model, a future glucose indication for the individual within the at least one future time period based on the one or more future glucose factors predicted and the at least one glucose measurement obtained for the individual; (v) provide, via a user device, the future glucose indication predicted for the individual.

2. The system of claim 1, wherein the one or more processors are further configured to perform the following operations:
(vi) generate a glucose intervention for the individual based on the future glucose indication predicted for the individual within the at least one future time period; and
(vii) provide, via the user device, the glucose intervention generated for the individual.

3. The system of claim 1, wherein the one or more data sources includes at least one of: a digital calendar of the individual; an email account; a social media account; and a text messaging account.

4. The system of claim 1, wherein the first trained model includes a natural language processor trained to extract future glucose factors from the contextual information received, the future glucose factors comprising anticipated meal consumption, anticipated physical activity, anticipated rest, anticipated alcohol consumption, and/or anticipated stress levels.

5. The system of claim 1, wherein the one or more future glucose indications predicted for the individual are conditioned on a likelihood of one or more future glucose factors predicted for the individual.

6. The system of claim 1, wherein the glucose measuring device is also the user device.

7. The system of claim 2, further comprising the user device configured to provide to the individual glucose interventions generated for the individual and future glucose indications predicted for the individual.

8. A method of predicting a future glucose indication of an individual, the method comprising:
obtaining, from one or more data sources, contextual information associated with the individual;
predicting, using a first trained model, one or more future glucose factors for the individual based on the contextual information received, wherein the one or more future glucose factors are relevant to at least one future time period;
obtaining, from a glucose measuring device, at least one current glucose measurement for the individual; and
predicting, using a second trained model, the future glucose indication for the at least one future time period based on the one or more future glucose factors predicted and the at least one current glucose measurement obtained for the individual.

9. The method of claim 8, wherein the one or more future glucose indications predicted for the individual are conditioned on a likelihood of one or more future glucose factors predicted for the individual.

10. The method of claim 8, wherein the one or more data sources includes at least one of: a photoplethysmography device; an electrocardiogram device; a galvanic skin response device; an accelerometer; a barometer; a gyroscope; a microphone; a camera; a global positioning system (GPS); and a thermometer.

11. The method of claim 8, wherein the one or more data sources includes at least one of: a digital calendar; an email account; a social media account; and a text messaging account.

12. The method of claim 8, wherein the first trained model includes a natural language processor trained to extract future glucose factors from the contextual information received, the future glucose factors comprising anticipated meal consumption, anticipated physical activity, anticipated rest, anticipated alcohol consumption, and/or anticipated stress levels.

13. The method of claim 8, wherein the one or more data sources includes a user input device, and the contextual information includes user input received via the user input device.

14. The method of claim 8, wherein the at least one future time period includes a time period encompassing between 1 and 2 hours in the future from a current time period.

15. The method of claim 8, wherein the second trained model includes a biophysical model, a transfer function, and/or an artificial intelligence model trained to predict one or more future glucose indications of a subject based on one or more current glucose level measurements obtained for the subject.
